(19) United States
(12) Patent Application Publication
(10) Pub. No.: US 2002/0198360 A1
(43) Pub. Date: Dec. 26, 2002

(54) STRUCTURAL MODELS FOR CYTOPLASMIC DOMAINS OF TRANSMEMBRANE RECEPTORS

(76) Inventors: MARK H. GINSBERG, SAN DIEGO, CA (US); MARTIN PFAFF, Seyssins (FR)

Correspondence Address: JANE MASSEY LICATA, LAW OFFICES OF JANE MASSEY LICATA, 66 E MAIN STREET, MARLTON, NJ, 08053

(21) Appl. No.: 09/320,907

(22) Filed: May 27, 1999

Related U.S. Application Data
(63) Continuation-in-part of application No. 09/187,236, filed on Nov. 5, 1998, now abandoned.

(51) Int. Cl. C12N 9/00; C07K 5/00; C07K 7/00; C07K 16/00; C07K 17/00; A61K 38/00; C07K 1/00; C07K 14/00; C08H 1/00

(52) U.S. Cl. 530/324; 530/350; 530/395; 530/402; 530/808

(57) ABSTRACT

Polypeptides containing a series of heptad-repeats that mimic a transmembrane domain and a selected cytoplasmic domain attached to the series of heptad repeats are provided which can be used in construction of structural models for evaluating the structure and activity of occupied, clustered and heteromeric transmembrane proteins and identifying therapeutic compounds.

### INTRODUCTION

This invention was made in the course of research sponsored by the National Institutes of Health. The U.S. Government may have certain rights in this invention.

This application is a continuation-in-part of U.S. patent application Ser. No. 09/187,236, filed Nov. 5, 1998.

### BACKGROUND OF THE INVENTION

In eukaryotic cells, many proteins extend through the cell membrane and therefore contain a cytoplasmic domain, a transmembrane domain and an extracellular domain. Many of these proteins are involved in signal transduction, cell adhesion and cell-cell interactions.

Among the proteins that fall into this category are the integrins. Integrins are involved in a number of pathological and physiological processes, including thrombosis, inflammation, and cancer. Other physiological and pathological conditions involving changes in cell adhesiveness are also mediated through integrins.

Many transmembrane proteins are oligomeric, being noncovalent associations of two or more different types of polypeptide subunits. In particular, integrins are heterodimers of two different protein subunits, designated α and β. The a subunits vary in size between 120 and 180 kDa and are each noncovalently associated with a β subunit. The extracellular domain of the integrin molecule forms a ligand binding site; both the α and β subunits are involved in forming the ligand binding site. A number of different ligands for integrins are known, including collagens, laminin, fibronectin, vitronectin, complement components, thrombospondin, and integral membrane proteins of the immunoglobulin superfamily such as ICAM-1, ICAM-2, and VCAM-1. The integrins recognize various short peptide sequences in their ligands. Examples of these are Arg-Gly-Asp (RGD), Lys-Gln-Ala-Gly-Asp-Val (KQAGDV; SEQ ID NO: 1), Asp-Gly-Glu-Ala (DGEA; SEQ ID NO: 2), and Glu-Ile-Leu-Asp-Val (EILDV; SEQ ID NO: 3). Variations in integrin function are often caused by changes in the ligand binding affinity of the extracellular domain of the integrins (J. S. Bennett & G. Vilaire J. Clin. Invest. 64:1393-1401 (1979); Altieri et al. J. Cell Biol. 107:1893-1900 (1988); Faull et al. J. Cell Biol. 121:155-162 (1993); Lollo et al. J. Biol. Chem. 268:21693-21700 (1993)).

Integrin α_{IIb}β₃ (platelet GPIIb-IIIa), a heterodimer of two type I transmembrane protein subunits, manifests highly regulated changes in ligand binding affinity. Affinity state-specific antibodies, e.g., PAC1 (Shattil et al. J. Biol. Chem. 260:1107-1114 (1985)), are useful for analysis of recombinant α_{IIb}β₃ in heterologous cells (O'Toole et al. Cell Regulation 1:883-893 (1990)). Platelet agonists increase the affinity of α_{IIb}β₃ (activation) probably by causing changes in the conformation of the extracellular domain (O'Toole et al. Cell Regulation 1:883-893 (1990); Sims et al. J. Biol. Chem. 266:7345-7352 (1991)). Cytoplasmic signaling pathways involving heterotrimeric GTP binding proteins, phospholipid metabolism, and serine-threonine kinases initiate these conformational changes in the extracellular domain; these changes may also involve calcium fluxes, tyrosine kinases, and low molecular weight GTP binding proteins (Sims et al. J. Biol. Chem. 266:7345-7352 (1991); Shattil et al. J. Biol. Chem. 267:18424-18431 (1992); S. J. Shattil & J. S. Brugge Curr. Opin. Cell Biol. 3:869-879 (1991); Ginsberg et al. Cold Spring Harbor Symposium of Quantitative Biology: The Cell Surface 57:221-231 (1992); Ginsberg et al. Curr. Opin. Cell Biol. 4:766-771 (1992); Nemoto et al. J. Biol. Chem. 267:20916-20920 (1992)). How cytoplasmic signals result in changes in the conformation and ligand binding affinity of the extracellular domain (“inside-out signal transduction”) of the integrin remains unknown. Studies with chimeras containing the cytoplasmic domains of various α and β subunits joined to the transmembrane and extracellular domain of α_{IIb}β₃ indicate that integrin cytoplasmic domains transduce cell type-specific signals that modulate ligand binding affinity. These signals require active cellular processes in both α and β cytoplasmic tails of the integrin, suggesting that they reflect physiologically relevant signals. In addition, deletion of a highly conserved motif, Gly-Phe-Phe-Lys-Arg (GFFKR; SEQ ID NO: 4), at the amino-terminus of the α subunit cytoplasmic domain, also resulted in high affinity binding of ligands to integrin α_{IIb}β₃. In contrast to the chimeras, high affinity ligand binding to GFFKR deletion mutants was independent of cellular metabolism, cell type, and the bulk of the β subunit cytoplasmic domain. Thus, integrin cytoplasmic tails are targets for the modulation of integrin affinity.

However, technical difficulties have greatly limited the application of high resolution techniques for determination of the structures of these proteins. In fact, molecular structures are available for only two intact transmembrane proteins, a bacterial photoreaction center (Deisenhofer et al. Nature 318:618-624 (1985)), and a porin (Weiss et al. FEBS Lett. 267:268-272 (1990)). Structures of receptor extracellular domains have been determined using soluble truncated extracellular domains as models (DeVos et al. Science 255:306-312 (1992); Milburn et al. Science 254:1342-1347 (1991)). These structures have contributed to the understanding of the basis of ligand recognition, but have provided less insight into the mechanism of signal transduction. Many membrane proteins that transduce signals are members of the Type I transmembrane protein family, the defining feature of which is a single membrane spanning region. These include the T cell receptor (A. Weiss Cell 73:209-212 (1993)); growth factor receptors (L. Patthy Cell 61:13-14 (1990)), and cytokine receptors (Miyajima et al. TIBS 17:378-382 (1992)). In general, the cytoplasmic domain of these proteins is critical for signaling. Thus, to understand signal transduction through such receptors, it is essential to understand the structure and function of the cytoplasmic domain. This is especially difficult for multisubunit Type I proteins.

A strategy for the chemical synthesis of structural models of the cytoplasmic domain of multisubunit transmembrane receptors has been previously proposed (Muir et al. Biochemistry 33:7701 (1994)). The cytoplasmic domains of integrin α_{IIb}β₃ were covalently linked via a helical coiled-coil made up of a series of identical heptad repeats. Coiled-coil tertiary structure was utilized to mimic the presumed helical membrane spanning domain and as a topological constraint, fixing the two integrin tails in a parallel orientation with the appropriate vertical stagger (Muir et al. Biochemistry 33:7701 (1994)). However, this synthetic approach poses limitations upon the polypeptide length and has a relatively modest yield.

Accordingly, there is a need for improved methods of producing structural models of the cytoplasmic domain of multisubunit transmembrane receptors. These models are useful in evaluating agents which control and modulate the activity of integrins and other transmembrane proteins, detecting their activity, and modulating their activity to detect and control physiological conditions.

### SUMMARY OF THE INVENTION

In the present invention, a method is provided for preparation of proteins for use in structural models or mimics of the cytoplasmic face of multimeric transmembrane proteins such as integrins. Proteins of the present invention may be prepared recombinantly or synthetically. However, by using recombinant proteins, limitations of polypeptide length and modest yield encountered in the initial synthetic approaches of the prior art are avoided. Accordingly, it is preferred that at least a portion of the structural model of the present invention be prepared recombinantly. In the model of the present invention, the heterodimeric nature of the β cytoplasmic domain is mimicked by use of covalent heterodimers of these domains. Helical coiled-coil architecture provides the desired parallel topology and vertical stagger of the tails. Further, it has been found that these proteins will spontaneously self-assemble into heteromeric structures when variant helical coiled-coil domains are used. The model is useful in studying protein interactions with transmembrane proteins such as integrin and screening agents for integrin inhibitory activity and in obtaining structures of integrin cytoplasmic domains. Heteromeric complexes and antibodies raised against these complexes can be used for structural analysis of cytoplasmic domains and to identify novel binding partners for heteromeric transmembrane proteins.

### BRIEF DESCRIPTION OF THE DRAWINGS

FIG. 1 exemplifies amino acid sequences of model proteins of integrin cytoplasmic domains. FIG. 1A shows the N-terminal (SEQ ID NO: 5) and heptad-repeat (SEQ ID NO: 6) structures common to all constructs. In the example shown, these are connected to the G1-β1A cytoplasmic domain (SEQ ID NO: 7). Arrows indicate the positions of hydrophobic residues corresponding to positions a and d of the heptad repeats. Positions of the additional Gly insertions in the G2-, G3- and G4-constructs are also indicated. FIG. 1B shows the integrin-specific sequences of the constructs used in experiments described herein including B1A (SEQ ID NO: 8), B1A (U788A) (SEQ ID NO: 9), B1B (SEQ ID NO: 10), B1C (SEQ ID NO: 11), B1D (SEQ ID NO: 12) and B7 (SEQ ID NO: 13). All integrin peptides correspond to the reported human integrin sequences.

FIG. 2 is a diagram of a mimic of the cytoplasmic domain of the transmembrane heterodimer, platelet αIIbβ3, synthesized with variant coiled-coil domains comprising GCN4 helices modified to contain residues to render the helices either fos-like or jun-like.

### DETAILED DESCRIPTION OF THE INVENTION

The present invention relates to the production of mimics of the cytoplasmic face of occupied and clustered transmembrane proteins such as integrins consisting of polypeptides comprising a series of a-helical heptad repeats, preferably 2 to 20, more preferably 3 to 6, most preferably 4, that mimic a transmembrane domain connected to a cytoplasmic domain of a selected multisubunit transmembrane receptors such as integrins. By “mimic” it is meant that the series of heptad repeats, imitates or replaces the structural features of the transmembrane domain. In one embodiment, an immobilizing epitope such as a His-Tag sequence or glutathione-S-transferase, is linked to the N-terminus for immobilization of the polypeptide in affinity chromatography. In this embodiment, it is preferred that the immobilizing epitope be linked to the polypeptide via a Cys-Gly linker. For convenience, a prokaryotic or chemical cleavage site such as a thrombin cleavage site can also be incorporated into the polypeptide at this linkage site.

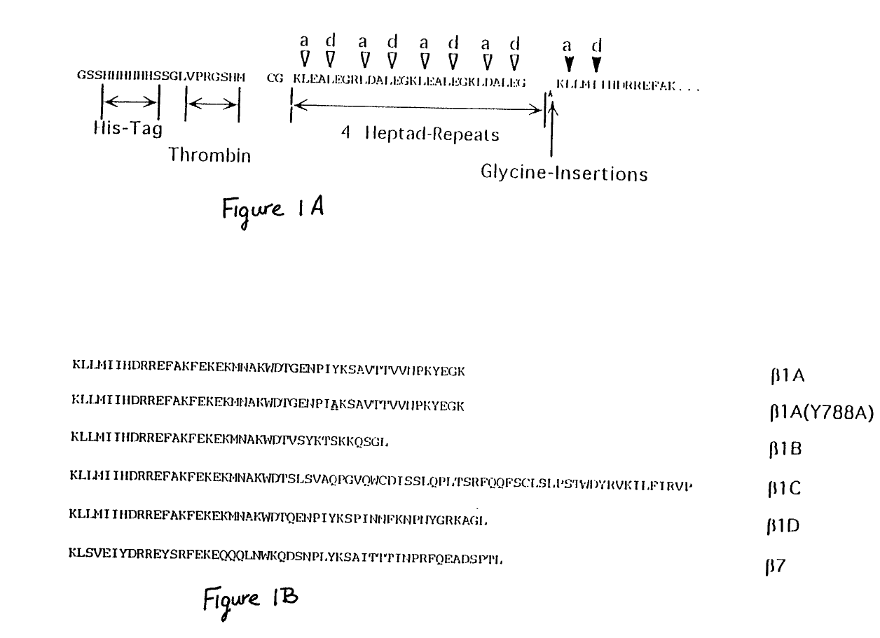

For the purposes of the present invention, by “α-helical heptad-repeat” it is meant a sequence consisting of substantially helical amphiphilic amino acids having hydrophobic residues at selected positions in the repeat, preferably positions a and d as depicted in FIG. 1. In such an embodiment, each repeat is seven amino acids with hydrophobic residues at the first and fourth positions. For example, in a preferred embodiment, the heptad repeat comprises the amino acid sequence G-X₁-L-X₂-X₃-L-X₄-G, (SEQ ID NO: 14) wherein X₁ is a lysine, arginine or ornithine, X₂ and X₄ are glutamic acid or aspartic acid, and X₃ is alanine, serine or threonine. The heptad repeats of the polypeptide are preferably identical. However, in some embodiments, each heptad repeat may differ in amino acid sequence. For example, it has been found that modifications to selected residues of the heptad repeat enhances formation of heteromeric structures. Thus, by the term “heptad repeats” it is also meant to include heptad repeats having at least one residue which has been modified to enhance formation of heteromeric structures. These heptad repeats are also referred to herein as “variant coiled-coil domains”. By “enhance” it is meant that the yield of heteromeric structures formed from the polypeptides is increased upon modification of one or more selected amino acids of the heptad repeat as compared to the yield of heteromeric structure formed by polypeptides with unmodified heptad repeats.

In a preferred embodiment, the cytoplasmic tail of a transmembrane receptor such as an integrin is linked to the heptad repeat via a glycine residue at the C-terminus of the heptad repeat. In this embodiment the polypeptide is predicted to form parallel coiled-coil dimers under physiological conditions. However, trimers and tetramers can also be designed based upon current methods for coiled coil protein design. These coiled-coil structures are likely to better mimic the proximity of transmembrane helices in the natural system and also ensure that a defined topology is maintained between the α and β cytoplasmic tails. In other words, the coiled-coil of the α-helical heptad repeat can act as a structural template onto which the cytoplasmic domain of the integrin or other transmembrane protein is attached. This ensures that the two cytoplasmic tails are staggered with respect to one another in a manner that approximates the intact protein. A cystine bridge ensures a parallel orientation and a correct stagger of the coiled-coil sequences within this dimer configuration. Examples of cytoplasmic tails of integrins which can be used include, but are not limited to which, β1A (SEQ ID NO: 8), β1A (Y788A) (SEQ ID NO: 9), β1B (SEQ ID NO: 10), β1C (SEQ ID NO: 11), β1B (SEQ ID NO: 12), β7 (SEQ ID NO: 13), β3 and αIIb.

It is preferred that at least a portion of the polypeptides used in the mimics of the present invention be prepared recombinantly. Recombinant preparation of polypeptides overcomes limitations of polypeptide length and modest yield encountered in the initial synthetic approaches of the prior art. Methods for recombinant preparation of at least a portion of a polypeptide are well known in the art. Polypeptides of the mimics or portions thereof may also be prepared synthetically. Methods for synthetic preparation of polypeptides are well known in the art. Further, methods for combining portions of synthetically and recombinantly prepared peptides into a single polypeptide are known. In the present invention, if both polypeptides of the mimic are prepared synthetically, at least one heptad repeat in the series of heptad repeats forming the coiled-coil sequences must differ in amino acid sequence from the other heptad repeats in the series. In a preferred embodiment, when both polypeptides of the mimic are prepared synthetically, the heptad repeats comprise variant coiled-coil domains.

Polypeptides of the model of the present invention are preferably >90% homogenous as determined by reverse phase C18 high pressure liquid chromatography and have a monomer mass that varies by less than 0.1% from that of the desired monomer sequence as determined by electrospray mass spectrometry. In this embodiment, formation of covalent dimers in aqueous solution can be observed by mass spectrometry and by SDS-PAGE, thus confirming the parallel orientation of the helices.

In this embodiment, the beginning of the integrin cytoplasmic domain sequence provides the hydrophobic residues of a fifth heptad repeat (FIG. 1). Consequently, direct linkage of the coiled-coil sequence of the α-helical heptad repeat could induce helical structure in the tail. To address this possibility, embodiments of the protein model containing additional glycines between the α-helical heptad repeats and the cytoplasmic domain sequence were synthesized (FIG. 1). Comparison was made of the CD-spectra of β1 integrin constructs containing either only one glycine (G1-β1A) or three additional glycines (G4-β1A) between the heptad repeats and the cytoplasmic domain. Insertion of glycines sharply reduced the minima at 208 and 222 nm. Consequently, predicted α-helical content in the protein model was reduced from 65% to 36%. The four heptad repeats constitute 27% of the mass of the construct; therefore, 36% helical content is consistent with the helical structure being limited to these repeats. Thus, the Gly insertion appears to eliminate α-helical structure induced in the cytoplasmic domain coiled-coil sequence.

To study possible influences of the structural changes induced by the Gly insertions, protein models were produced having the β1A cytoplasmic domain with one, two and three additional Gly residues inserted after the heptad-repeat motif (G2-, G3-, G4-β1A) and compared with the G1-β1A construct. As an additional control, a variant of the G4-β1A peptide was produced with a Tyr to Ala substitution in the membrane-proximal NPXY-motif (G4-β1A-Y788A) (FIG. 1). This mutation interferes with focal adhesion targeting and activation of integrins. The purified proteins were bound via their N=terminal His-Tag to a Ni²⁺⁻ resin and used in affinity chromatography experiments with lysates of NHS-biotin-labeled human platelets. Marked changes in the pattern of protein binding were observed as a consequence of the Gly insertions. Polypeptides migrating at 45, 56, 58, 140 and 240 kDa bound only to the mimics with Gly insertions. The Y788A mutation in the G4-β1A construct (YA) suppressed the interaction with the 240 kDa, but not with the other components. Using monoclonal antibodies, the 240 kDa and 45 kDa proteins were identified as filamin and actin, respectively. The enriched 56, 58 and 140 kDa polypeptides have not been identified but have failed to react with antibodies specific for pp60^{src}, paxillin, ppl25^{fak}, α-actinin, vinculin and pp72^{syk} in Western blotting experiments. Talin bound to the G1- and G4-β1A construct but not to the Y788A-G4β1A construct. Thus, the structural changes in the model induced by the insertion of glycines into the coiled-coil motif and the integrin cytoplasmic domain sequence alter interactions of these proteins with cellular components. Alterations of the β1A tail that block cytoskeletal interactions, such as the Y788 mutation and β1B- and β1C-splice variants also abrogate binding to talin and filamin. Consequently, the observed in vitro interactions are likely to be biologically relevant.

Models of the present invention were also constructed with G1- and G4- polypeptides of the muscle-specific splice variant β1D and the β7 integrin subunits (FIG. 1) to study binding interactions of various integrin binding proteins. When used with NHS-biotinylated platelet lysates, the β1D constructs bound more talin and β7 constructs bound more filamin, compared to β1A. In addition, these differences in binding were consistently observed when lysates of a human T-cell leukemia cell line (Jurkat) , a human fibrosarcoma cell line (HT 1080), and a differentiated myotubes derived from a mouse myoblast cell line (C2C12) , were used for affinity-chromatography. Moreover, stronger binding of the β1D constructs to talin and of the β7 constructs to filamin was independently observed, both with the G1- as well as the G4- variants of the model proteins, indicating that the structural changes induced by Gly insertions do not strongly influence these differential interactions.

Purified preparations of these proteins were then used to demonstrate that the observed interactions with talin and filamin in the cell extracts are direct. The relative amounts of purified filamin and talin bound to the model proteins were similar to those observed with cell lysates. Specifically, β1D constructs bound more talin and β7 constructs bound more filamin than β1A protein models. In addition, binding of both cytoskeletal proteins to the G4-Y788A-β1A construct and to the G4-β1B and G4-β1C variants was functionally reduced compared to G4-β1A. Moreover, G4-constructs of β1A, β1D and β7 integrin cytoplasmic domains bound more purified filamin than the corresponding G1-constructs. However, the G1-β7 model protein still bound more filamin than G4-β1A or G4-β1D. A densitometric evaluation of the Coomassie blue-stained gels indicated that the β1D construct bound about nine times more talin, and the β7 construct bound 8.4 times more filamin than the β1A model protein. In these experiments, there was a >10 fold molar excess of model proteins relative to the quantity of talin and filamin. Thus, the affinity of β1A for filamin is at least eight fold less than that of β7, and its affinity for talin is at least nine fold less than that of β1D.

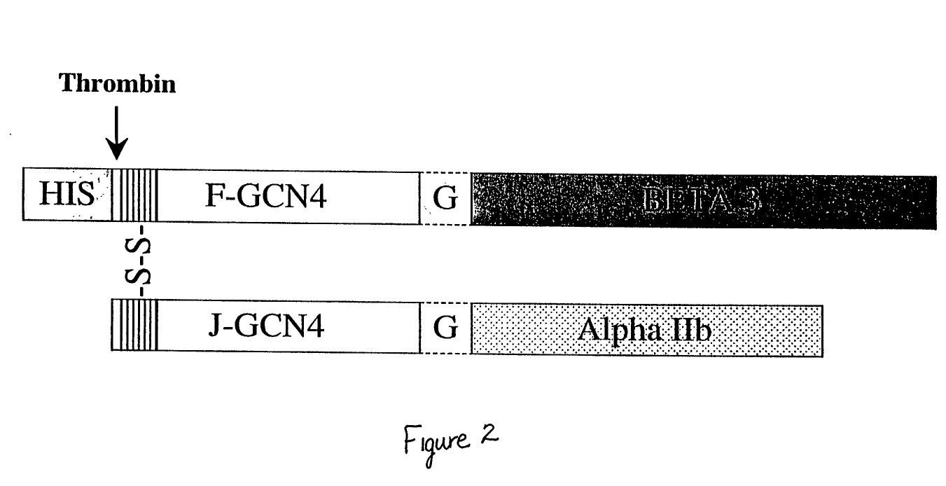

Using variant coiled-coil domains of heptad repeats having modified amino acid residues, polypeptides have also been prepared that preferentially form heteromeric structures. For example, as depicted in FIG. 2, a mimic of the cytoplasmic domain of the transmembrane heterodimer, platelet αIIbβ3 (GPIIb-IIIa) was produced. This was done by use of a GCN4 helix modified to contain two residue substitutions to make it fos-like or with substitutions to make it jun-like. The cytoplasmic domain of the β₃ subunit was joined to the fos-like helix while that of α_{IIb} was joined to the jun-like helix (John et al. J. Biol. Chem. 269:16247-16253 (1994)). In addition, as diagramed in FIG. 2, this construct may further comprise an N-terminal HIS tag on the β3 subunit that is useful in immobilization for affinity chromatography. The two subunits or proteins spontaneously self-assemble into heterodimers. Using a non-reduced gel, it was confirmed that in this embodiment, all protein is a heterodimer. Reduction resulted in separation of the heterodimer into the individual proteins or subunits. Ion spray mass spectroscopy was used to confirm the mass of this heterodimer. These modifications to the heptad repeat of the polypeptides nearly doubled the yield of heterodimer formed as compared to heterodimer formed by polypeptides with unmodified heptad repeats.

Antibodies against these heteromeric complexes of the present invention that recognize combinatorial epitopes of the cytoplasmic domains of the complex can be produced in accordance with well known techniques. For example, antibodies were prepared against the heterodimeric complex of FIG. 2. It was found that this antibody raised against this synthetic mimic of the present invention reacted with the native transmembrane protein, platelet α_{IIb}β₃. However, this reactivity could be completely blocked by addition of the mimic. Reactivity was not inhibited by the full-length α_{IIb} peptide and only partially inhibited by the full-length β₃ peptide. However, a mixture of the two linear peptides together produced complete inhibition. Thus, this antibody recognized combinatorial epitopes in the cytoplasmic domain of the native receptor that were mimicked in the model protein.

The combinatorial epitopes are also manifest in a mutant of the β3 domain. This mutant, β3(Y747A), is known to profoundly disrupt the function of the β₃ cytoplasmic domain leading to a failure of both inside-out and outside-in integrin signaling. Unlike wild-type β₃, the β3(Y747A) mutant completely inhibited binding of the antibody to platelet α_{IIb}β₃. Further, addition of the α_{IIb} peptide resulted in no change in inhibition by this mutant. Thus, it appears that the interaction of the α_{IIb} and β₃ subunit results in a change in the conformation of the β₃ cytoplasmic domain which inhibits its signaling function. Further, small molecules that bind to the β₃ cytoplasmic domain and induce this conformational change will also inhibit signaling through this integrin.

Antibodies raised against a mimic of the present invention were used in an immunochemical assay to identify compounds that bind to the β₃ cytoplasmic domain and induce a conformation change. In this assay, the binding of the α_{IIb} and β₃ cytoplasmic tails was first analyzed. The apparent affinity of the interaction was high. Further, it was specific since the cytoplasmic domains of α4 and α5 lack this effect. It was also found that the β3 binding motif in α_{IIb} was localized to a heptapeptide (See Table 1). Furthermore, as shown in this Table, point mutations or deletions that disrupted this heptapeptide sequence in the α_{IIb} cytoplasmic domain also disrupted interaction with β₃.

TABLE 1

| Peptide | EC50 (nM) | SEQ ID NO |
|---|---|---|
| KVGFFKRNRPPLEEDDEEGQ | 3 | 15 |
| KVGFFKRNRPPLEEDD | 5 | 16 |
| KVGFFKRNRPPLEE | >1000 | 17 |
| KRNRPPLEEDDEEGQ | 6 | 18 |
| NRPPLEEDDEEGQ | 8 | 19 |
| PLEEDDEEGQ | >1000 | 20 |
| NRPPLEEDD | 20 | 21 |
| RPPLEED | 30 | 22 |
| PPLEED | >1000 | 23 |
| KVGFFKRNRPPLEEAAEEGQ | >1000 | 24 |
| KVGFFPLEEAAEEGQ | >1000 | 25 |

Thus, these experiments confirm the utility of this immunochemical assay in structure/function analysis for integrin β₃ cytoplasmic domain binding partners. Based upon the nature of the peptide sequence in α_{IIb} that initiates the β₃ conformation, small molecule mimics of this disclosed peptide sequence or derived from random screening using this immunoassay can be identified as anti-thrombotic agents.

As demonstrated by these experiments, the structural models of the present invention provide a novel experimental tool for the analysis of various proteins associations with integrin tails in vitro and the structural aspect of the cytoplasmic face of integrins. The structural models of the present invention thus have a number of applications based upon their ability to maintain the cytoplasmic tails of the construct in a configuration that is equivalent or similar to the configuration predominating in vivo while maintaining solubility and stability in an aqueous system, namely in staggered, parallel, and proximal topology. For example, these models can be used to detect intracellular molecules capable of binding to integrins and modulating their affinity by inside-out signaling. Alternatively, these molecules can be used in vivo to disrupt or modulate inside-out signaling by binding to the cells in a manner such that the cytoplasmic domains of these models compete for intracellular molecules with the natural integrins. Because these structural models do not contain the extracellular ligand-binding sites of integrins, they would then disrupt inside-out signaling. This would be particularly useful in conditions in which overactivity of integrins is involved, such as inflammation, thrombosis, and malignancy. This would provide a new method of treating such conditions or their sequelae; because these molecules mimic the orientation of the natural integrins within the membrane, they would not disrupt membrane structure and would therefore be better tolerated and avoid side effects. Additionally, structural models of the present invention can be used to detect molecules capable of binding to the intracellular or cytoplasmic domain of integrins and other transmembrane molecules in vivo, such as by affinity chromatography. Alternatively, molecules can be identified as cytoplasmic domain binding partners by measuring binding of an antibody raised against a heteromeric complex to native transmembrane receptor in the presence and absence of the molecule. A change in binding of the antibody to the native transmembrane receptor in the presence of the molecule is indicative of the molecule being a cytoplasmic domain binding partner. Accordingly, these models are useful in identifying various therapeutic compounds for selected cytoplasmic domains. By “therapeutic compounds” it is meant to include, but is not limited to, molecules which are found to bind to a selected cytoplasmic domain of the model, molecules which bind to proteins that bind to the cytoplasmic domain of the model, and the models themselves.

The following examples are provided for illustrative purposes only and are not intended to limit the invention.

### EXAMPLES

### Example 1

### Antibodies and cDNAs

Antibodies for the analysis of proteins bound to cytoplasmic domain model proteins on Western blots included: goat serum against filamin (Sigma Chemical Co., St. Louis, Mo.), rabbit serum against α-actinin (Sigma Chemical Co.), mAbs against talin (clone 8d4) (Sigma Chemical Co.), vinculin (clone hVIN-1) (Sigma Chemical Co.), pacillin (clone Z035) (Zymed Laboratories Inc., S. San Francisco, Calif.), filamin (MAB1680) (Chemicon International Inc. Temecula, Calif.), α-actinin (MB75.2) (Sigma Chemical Co.), actin (clone C4) (Boehringer-Mannheim Corp., Indianapolis, Ind.), mAb against pp60_{src} (clone 327), polyclonal rabbit serum against pp^{125FAK} (BC3) and rabbit anti-pp72^{syk}.

Antibodies to the model protein depicted in FIG. 2 were raised in 2.5 kg New Zealand white rabbits. One hundred microliters of a water solution containing 1 mg/ml of the model protein was emulsified in 1 ml incomplete Freund's adjuvant and administered subcutaneously to each New Zealand white rabbit. Two additional injections at 2-week intervals were followed by bleeding (approximately 50 mls) at monthly intervals until the rabbits were sacrificed. Blood was permitted to clot at room temperature, and the serum was recovered by centrifugation. Serum was heat inactivated at 56° C. for 30 minutes and stored at −20° C. A pre-immune serum, obtained prior to immunization, was used as a control.

Human cDNA used in these experiments included: β1C cDNA; β1 cDNA with the point mutation, Y788Al; a cDNA for the cytoplasmic domain of human integrin β1D obtained by RT-PCT of heart muscle total RNA; cDNA of human integrin β7; and a cDNA coding for the human β1B subunit cytoplasmic domain synthesized in PCR reactions using a human β1A vector with a partially overlapping reverse-oligonucleotide containing the human β1B sequence.

### Example 2

### Recombinant Cytoplasmic Domain Models

Oligonucleotides were synthesized and used in PCR reactions to create a cDNA for the α-helical heptad repeat protein sequence KLEALEGRLDALEGKLEALEGKLDALEG (SEQ ID NO: 6) G1-([heptad]₄). Variants containing 1 to 3 additional Gly residues (G2-4-([heptad]₄)) at the C-terminus were synthesized by modification of the antisense oligonucleotide. These cDNAs were ligated into a NdeI-HindIII restricted modified pET15b vector (Novagen, Madison, Wis.). Integrin cytoplasmic domains were joined to the helix as a HindIII-BamHI fragments. The final constructs coded for the N-terminal sequence GSSHHHHHHSSGLVPRGSHMCG (SEQ ID NO: 5) [heptad]₄ linked to the cytoplasmic domains of integrins. Different cytoplasmic domain cDNAs were cloned via PCR from appropriate cDNAs using forward oligonucleotides introducing a 5′-HindIII site and reverse oligonucleotide creating a 3′-BamHI site directly after the Stop-codon. PCR products were first ligated into the pCR™ vector using the TA cloning® kit (Invitrogen Corp., San Diego, Calif.) . After sequencing, HindIII/BamHI inserts were ligated into a modified pET15b vector. Recombinant expression in BL21(DE3)pLysS cells (Novagen) and purification of the recombinant products were performed according to the pET System Manual (Novagen) with an additional final purification step on a reverse phase C18 HPLC column (Vydac, Hesperia, Calif.). Products were analyzed by electrospray mass spectrometry on an API-III quadruple spectrometer (Sciex, Toronto, Ontario, Canada).

### Example 3

### Ultraviolet Circular Dichroism Spectroscopy

Far UV CD spectra were recorded on an AVIV 60DS spectropolarimeter with peptides dissolved in 50 mM boric acid pH 7.0. Data were corrected for the spectrum obtained with buffer only and related to protein concentrations determined from identical samples by quantitative amino acid analysis. From these values, the percentage of helical secondary structure was calculated in accordance with procedures described by Muir et al. Biochemistry 33:7701 (1994).

### Example 4

### Cells and Cell Lysates

Human platelets were obtained by centrifugation of freshly drawn blood samples at 1000 rpm for 20 minutes and sedimentation of the resulting platelet-rich plasma at 2600 rpm for 15 minutes. They were washed twice with 0.12 M NaCl, 0.0129 M trisodium citrate, 0.03 M glucose, pH 6.5, and once in Hepes-Saline (3.8 mM Hepes, 137 mM NaCl, 2.7 mM KCl, 5.6 mM D-Glucose, 3.3 mM Na₂HPO₄, pH 7.3-7.4). Human Jurkat and HT1080 cells and mouse C2C12 cells were obtained from the American Type Culture Collection (Rockville, Md.) and cultured in RPMI1680 (Jurkat) or DMEM with 10% fetal calf serum. For differentiation to myotubes, C2C12 myoblasts were kept confluent in DMEM with 5% horse serum for 6 days. Cultured cells were washed twice in phosphate-buffered saline (PBS) and biotinylated with 1 mM NHS-biotin (Pierce) in PBS during 30 minutes at room temperature. Platelets were biotinylated in Hepes-Saline. After two additional washes with TBS, cells were lysed on ice with buffer A (1 mM Na₃VO₄, 50 mM NaF, 40 mM NaPyrophosphate, 10 mM Pipes, 50 mM NaCl, 150 mM sucrose, pH 6.8) containing 1% TRITON X-100, 0.5% sodium deoxycholate, 1 mM EDTA and protease inhibitors (1/100th volume of aprotinin (Sigma A-6379), 5 μg/ml leupeptin, 1 mM PMSF). To platelet lysates 0.1 mM of the calpain inhibitor E-64 (Boehringer Mannheim) were added in addition. Lysates were sonicated 5 times on ice for 10 seconds at a setting of 3 using an Astrason Ultrasonic Processor (Heart Systems, Farmingdale, N.Y.). After 30 minutes, lysates were clarified by centrifugation at 12,000 g for 30 minutes.

### Example 5

### Affinity Chromatography Experiments with Integrin Cytoplasmic Domain Mimics

Purified recombinant cytoplasmic domain proteins (500 μg) were dissolved in a mixture of 5 ml 20 mM Pipes, 50 mM NaCl, pH 6.8 and 1 ml 0.1 M sodium acetate, pH 3.5 and bound overnight to 80 μl of Ni²⁺ saturated His-bind resin (Novagen). In control experiments, it was found that this leads to approximate saturation of the resin with peptide. Resins were washed twice with 20 mM Pipes, 50 mM NaCl, pH 6.8, and stored at 4° C. with 0.1% sodium azide as suspensions with one volume of this buffer. Fifty microliters of such a suspension were added to 4.5 ml of cell lysates which had been diluted tenfold with buffer A containing 0.05% TRITON X-100, 3 mM MgCl₂ and protease-inhibitors. After incubation overnight at 4° C., resins were washed five times with this buffer and finally heated in 50 μl of reducing sample buffer for SDS PAGE. Samples were separated on 4-20% SDS polyacrylamide gels (NOVEX) and either stained with Coomassie or transferred to Immobilon P membranes (Amersham Corp., Arlington Hts, Ill.). Membranes were blocked with TBS, 5% nonfat-mild powder and stained with streptavidin-peroxidase (VECTASTAIN) or specific antibodies. Bound peroxidase was detected with an enhanced chemiluminescence kit (Amersham).

### Example 6

### Binding to Purified Talin and Filamin

Human uterus filamin (ABP-280) was prepared as a 1.5 mg/ml solution in 0.6 M KCl, 0.5 mM ATP, 0.5 mM DTT, 10 mM imidazole, pH 7.5. For binding assays performed as described in Example 5, this solution was diluted 1/12 with buffer A, 0.05% TRITON X-100, 3 mM MgCl₂, 2 mg/ml BSA, protease-inhibitors (see Example 5), omitting the 50 mM NaCl (see Example 5), and resins with bound model proteins were added. Washing was performed in this buffer without BSA and with additional 50 mM KCl.

Talin was purified from human platelets in accordance with well known procedures with an additional purification step using chromatography on phosphocellulose and stored at 1 mg/ml in 10 mM NaCl, 50% glycerol. This solution was diluted to either 87 or 17 μg/ml talin with buffer A, 0.05% TRITON X-100, 3 mM MgCl₂, 2 mg/ml BSA and protease inhibitors (see Example 5, including 0.1 mM E-64) and processed as indicated in the binding assays with cell lysates. For densitometric analysis, scans of Coomassie-stained gels were processed using the program NIH-Image (NIH, Bethesda, Md.). Equal loading of gels was controlled in Coomassie-stained gels of the recombinant cytoplasmic domain polypeptides coeluted with the ligand from the resins.

### Example 7

### EC50 Determination

αIIbβ3 was purified by gel filtration in accordance with procedures described by Du et al. (Cell 65:409-416 (1991)) with omission of the heparin and Con A affinity chromatography steps. The final product was greater than 95% homogenous as judged by SDS PAGE. In the enzyme-linked immunosorbent assay (ELISA) the αIIbβ3 was used at a concentration of 5 βg/ml in a coating buffer containing 0.1 M NaHCO₃ and 0.05% NaN₃ . Fifty ul/well was used to coat IMMULON II microtiter wells at 4° C. overnight. After removal of the coating solution, 150 μl blocking buffer(coating buffer containing 5% bovine serum albumin) was added. After an additional one hour incubation at 4° C., the blocking buffer was removed and the plates were washed three times with wash buffer (0.01 M Tris, 0.15 M NaCl, 0.01% thimerisol, 0.05% Tween 20, pH 8.0). Twenty-five microliters of the competitor was added to each well followed by 25 μl of a dilution of the anti-model protein antibody. Following mixing, the plate was covered for one hour at 37° C. and washed four times with wash buffer. To quantify bound antibody, 50 μl of horseradish peroxidase-conjugated goat anti-rabbit IgG (Biorad) was diluted to a concentration of 1/1000 in wash buffer containing 1 mg/ml bovine serum albumin. These plates were then incubated at 37° C. for 1.5 hours. After four washes, bound antibody was assayed by measuring peroxidase activity with O-phenylenediamine as a substrate and quantifying reaction product by its optical density at 490 nm. Data were expressed as B/B0 where B=A₄₉₀ in the presence of competitor and B0=A₄₉₀ in its absence. In some experiments, varying concentrations of αIIb peptides were added to a fixed, saturating, quantity of β3 peptide (20-50 nM). Competition was again expressed as B/B0, however, B0 was the A₄₉₀ in the presence of the β3 peptide and no added αIIb peptide. EC50 was defined as the dose of αIIb resulting in B/B0=0.5.

What is claimed is:

1. A polypeptide comprising:
(a) a series of heptad-repeats that mimic a transmembrane domain; and
(b) a selected cytoplasmic domain attached to the heptad repeats,
wherein at least a portion of the polypeptide is prepared recombinantly.

2. The polypeptide of claim 1 wherein the selected cytoplasmic domain is an integrin cytoplasmic domain.

3. The polypeptide of claim 2 wherein the integrin cytoplasmic domain is selected from a group consisting of β1A, β1B, β1C, β1D, β7, αIIb and β3.

4. The polypeptide of claim 1 further comprising one or more glycine residues inserted between the heptad repeats and the selected cytoplasmic domain.

5. The polypeptide of claim 1 further comprising an immobilizing epitope linked to the series of heptad repeats of the polypeptide via a Cys-Gly linker.

6. The polypeptide of claim 5 wherein a chemical or prokaryotic cleavage site is inserted between the immobilizing epitope and the Cys-Gly linker.

7. A structural model of a selected cytoplasmic domain comprising a polypeptide of claim 1 for evaluating structure and activity of a selected occupied and clustered transmembrane protein having the selected cytoplasmic domain.

8. A structural model of a selected cytoplasmic domain comprising a polypeptide of claim 4 for evaluating structure and activity of a selected occupied and clustered transmembrane protein having the selected cytoplasmic domain.

9. A structural model of a selected cytoplasmic domain comprising a polypeptide of claim 1 for use in identification of therapeutic compounds.

10. A heteromeric complex comprising at least two polypeptides of claim 1.

11. The polypeptide of claim 1 wherein at least one residue of the series of heptad-repeats is modified to enhance formation of heteromeric structures which mimics a cytoplasmic domain of a heteromeric transmembrane receptor.

12. A heteromeric complex comprising at least two polypeptides of claim 11 which mimics a cytoplasmic domain of a heteromeric transmembrane receptor.

13. The heteromeric complex of claim 12 wherein a first polypeptide comprises a GCN4 helix modified to contain two residue substitutions to make it fos-like joined to a β3 subunit and a second polypeptide comprises a GCN4 helix modified to contain two residue substitutions to make it jun-like joined to an α_{IIb} subunit, said heteromeric complex mimicking platelet α_{IIb}β₃.

14. An antibody raised against the heteromeric complex of claim 12.

15. A method for identifying a cytoplasmic domain binding partner comprising measuring binding of the antibody of claim 14 to native transmembrane receptor in the presence and absence of a molecule wherein a decrease in binding of the antibody to the native transmembrane receptor in the presence of the molecule is indicative of the molecule being a cytoplasmic domain binding partner.

16. A polypeptide comprising:
(a) a series of heptad-repeats that mimic a transmembrane domain; and
(b) a selected cytoplasmic domain attached to the heptad repeats,
wherein at least one heptad repeat in the series has a different amino acid sequence to other heptad repeats in the series.

17. A heteromeric complex comprising at least two polypeptides of claim 16.

18. The polypeptide of claim 16 wherein at least one residue of the series of heptad-repeats is modified to enhance formation of heteromeric structures which mimics a cytoplasmic domain of a heteromeric transmembrane receptor.

19. A heteromeric complex comprising at least two polypeptides of claim 18.


SEQUENCE LISTING (25 sequences)

SEQ ID NO: 1 - protein, 6 aa, Artificial Sequence
  Description of Artificial SequenceSynthetic
KQAGDV

SEQ ID NO: 2 - protein, 4 aa, Artificial Sequence
  Description of Artificial SequenceSynthetic
DGEA

SEQ ID NO: 3 - protein, 5 aa, Artificial Sequence
  Description of Artificial SequenceSynthetic
EILDV

SEQ ID NO: 4 - protein, 5 aa, Artificial Sequence
  Description of Artificial SequenceSynthetic
GFFKR

SEQ ID NO: 5 - protein, 20 aa, Artificial Sequence
  Description of Artificial SequenceSynthetic
GSSHHHHHHSSGLVPRGSHM

SEQ ID NO: 6 - protein, 28 aa, Artificial Sequence
  Description of Artificial SequenceSynthetic
KLEALEGRLDALEGKLEALEGKLDALEG

SEQ ID NO: 7 - protein, 14 aa, Artificial Sequence
  Description of Artificial SequenceSynthetic
KLLMIIHDRREFAK

SEQ ID NO: 8 - protein, 47 aa, Artificial Sequence
  Description of Artificial SequenceSynthetic
KLLMIIHDRREFAKFEKEKMNAKWDTGENPIYKSAVTTVVNPKYEGK

SEQ ID NO: 9 - protein, 47 aa, Artificial Sequence
  Description of Artificial Sequence Synthetic
KLLMIIHDRREFAKFEKEKMNAKWDTGENPIAKSAVTTVVNPKYEGK

SEQ ID NO: 10 - protein, 38 aa, Artificial Sequence
  Description of Artificial SequenceSynthetic
KLLMIIHDRREFAKFEKEKMNAKWDTVSYKTSKKQSGL

SEQ ID NO: 11 - protein, 74 aa, Artificial Sequence
  Description of Artificial SequenceSynthetic
KLLMIIHDRREFAKFEKEKMNAKWDTSLSVAQPGVQWCDISSLQPLTSRFQQFSCLSLPSTWDYRVKILFIRVP

SEQ ID NO: 12 - protein, 50 aa, Artificial Sequence
  Description of Artificial SequenceSynthetic
KLLMIIHDRREFAKFEKEKMNAKWDTQENPIYKSPINNFKNPNYGRKAGL

SEQ ID NO: 13 - protein, 52 aa, Artificial Sequence
  Description of Artificial SequenceSynthetic
KLSVEIYDRREYSRFEKEQQQLNWKQDSNPLYKSAITTTINPRFQEADSPTL

SEQ ID NO: 14 - protein, 8 aa, Artificial Sequence
  Description of Artificial SequenceSynthetic
GXLXXLXG

SEQ ID NO: 15 - protein, 20 aa, Artificial Sequence
  Description of Artificial SequenceSynthetic
KVGFFKRNRPPLEEDDEEGQ

SEQ ID NO: 16 - protein, 16 aa, Artificial Sequence
  Description of Artificial SequenceSynthetic
KVGFFKRNRPPLEEDD

SEQ ID NO: 17 - protein, 14 aa, Artificial Sequence
  Description of Artificial SequenceSynthetic
KVGFFKRNRPPLEE

SEQ ID NO: 18 - protein, 15 aa, Artificial Sequence
  Description of Artificial SequenceSynthetic
KRNRPPLEEDDEEGQ

SEQ ID NO: 19 - protein, 13 aa, Artificial Sequence
  Description of Artificial SequenceSynthetic
NRPPLEEDDEEGQ

SEQ ID NO: 20 - protein, 10 aa, Artificial Sequence
  Description of Artificial SequenceSynthetic
PLEEDDEEGQ

SEQ ID NO: 21 - protein, 9 aa, Artificial Sequence
  Description of Artificial SequenceSynthetic
NRPPLEEDD

SEQ ID NO: 22 - protein, 7 aa, Artificial Sequence
  Description of Artificial SequenceSynthetic
RPPLEED

SEQ ID NO: 23 - protein, 6 aa, Artificial Sequence
  Description of Artificial SequenceSynthetic
PPLEED

SEQ ID NO: 24 - protein, 20 aa, Artificial Sequence
  Description of Artificial SequenceSynthetic
KVGFFKRNRPPLEEAAEEGQ

SEQ ID NO: 25 - protein, 15 aa, Artificial Sequence
  Description of Artificial SequenceSynthetic
KVGFFPLEEAAEEGQ